(19) United States
(12) Patent Application Publication
(10) Pub. No.: US 2008/0221458 A1
(43) Pub. Date: Sep. 11, 2008

(54) Catheter and method for diagnosis and treatment of diseased vessels

(76) Inventors: Robert W. Scott, Indianapolis, IN (US); Steven J. Rychnovsky, Santa Barbara, CA (US); Ian M. Leitch, Goleta, CA (US); Jeffrey A. Vasek, Santa Barbara, CA (US); John A. Franco, Emeryville, CA (US)

Correspondence Address: BRYAN CAVE LLP, 211 NORTH BROADWAY, SUITE 3600, ST. LOUIS, MO, 63102-2750, US

(21) Appl. No.: 12/152,612

(22) Filed: May 15, 2008

Related U.S. Application Data
(63) Continuation of application No. 10/634,665, filed on Aug. 5, 2003, now abandoned.
(60) Provisional application No. 60/401,063, filed on Aug. 5, 2002.
(60) Provisional application No. 60/401,065, filed on Aug. 5, 2002.

(51) Int. Cl. A61B 6/00

(52) U.S. Cl. 600/478

(57) ABSTRACT

The present invention provides a catheter for detecting and treating diseased tissue in a blood vessel or other hollow body organ. The catheter comprises an elongated tubular catheter shaft having a distal end comprising a light transmission zone. A first fiber lumen in the catheter shaft contains a diagnostic optical fiber having a distal end terminating within the light transmission zone for emitting and receiving light through the light transmission zone. A diagnostic subassembly at the proximal end and in communication with the diagnostic optical fiber processes diagnostic light for use in connection with a diagnostic method for detecting diseased tissue. A second fiber lumen can be provided in the catheter shaft for containing a treatment optical fiber for delivering treatment light from a light source at the proximal end of the catheter shaft to the light transmission zone. The treatment optical fiber has a distal end terminating within the light transmission zone for emitting light for treatment of the diseased tissue. An occlusion balloon is positioned on the distal end of the catheter shaft adjacent to the light transmission zone and in fluid communication with an inflation lumen. One or more infusion ports formed on or near the light transmission zone and in fluid communication with an infusion lumen deliver infusion fluid to the hollow body organ.

### CROSS-REFERENCE TO RELATED APPLICATIONS

This application is a continuation of and claims priority to U.S. application Ser. No. 10/634,665, filed Aug. 5, 2003, which claims priority to U.S. Provisional Application No. 60/401,063, filed Aug. 5, 2002, and U.S. Provisional Application No. 60/401,065, filed Aug. 5, 2002.

### FIELD OF THE INVENTION

The invention relates to the field of medical instruments used in diagnosing diseased conditions and administering light for therapeutic methods, such as photodynamic therapy (PDT). The present invention provides a catheter for detecting and treating diseased tissue in a blood vessel or other hollow body organ, which effectively eliminates blood from the light transmission site to improve diagnostic and treatment functions.

### BACKGROUND

Historically, a primary concern in cardiovascular disease indications, such as atherosclerosis and restenosis, has been the identification and treatment of partial or total occlusions within vessels. The standard diagnostic tool for identifying such occlusions is angiography. Recent research in the cardiovascular area has determined that certain types of lesions known as vulnerable plaques (VP) may be responsible for a significant portion of sudden cardiac related deaths. Unfortunately, in most cases, VP lesions cannot be diagnosed by angiography. This has led to the development of several catheter-based diagnostic technologies for identification of such cardiovascular conditions as vulnerable plaques, inflammation and atherosclerosis that are not always detectable with angiography. These diagnostic technologies include optical coherence tomography (OCT), fluorescence detection (FD), active light detection (such as, reflectance spectroscopy using visible or infrared (IR) light), and passive IR detection (similar to thermal imaging).

One problem with each of these techniques is that the presence of blood within the vessel can impede the performance of the diagnostic. Another drawback of these technologies is the potential for error when attempting to treat a target site identified with a diagnostic catheter. For example, the conventional method for identifying and treating VP generally involves positioning a diagnostic catheter within a blood vessel such that the diagnostic element can be moved through the vessel in a scanning procedure to locate VP lesions. If a VP lesion is identified, its location is noted, after which the vessel is further scanned for other VP lesions. Once this scanning is complete, the diagnostic catheter is removed and replaced with a treatment catheter, which is positioned at each previously located VP lesion to allow the treatment to be performed, for example, by catheter-based photodynamic therapy (PDT).

The approach outlined above presents several problems. First, this approach requires two separate catheters which add to the expense of the procedure. Second, in practice it is difficult to accurately reposition the treatment catheter at the various sites originally identified by the diagnostic catheter. This can result in the treatment being delivered at a site different from that identified by the diagnostic catheter, a condition referred to as geographic mismatch. Finally, the above approach lacks convenience and extends the overall time of the procedure.

Thus, there is a need for a catheter that provides an effective means for both diagnosis and treatment of diseased tissue within blood vessels and other hollow body organs. The integrated diagnosis and treatment catheter and method disclosed herein provides these means, thereby avoiding the limitations of prior devices and methods outlined above.

### SUMMARY

The present invention provides a catheter for detecting and treating diseased tissue in a blood vessel or other hollow body organ. The catheter comprises an elongated tubular catheter shaft having a proximal end which remains outside of the body organ when in use and a distal end which is inserted into the body organ when in use. The distal end has a light transmission zone through which light can be transmitted. A first fiber lumen in the catheter shaft contains a diagnostic optical fiber having a distal end terminating within the light transmission zone for emitting and/or receiving light through the light transmission zone. A diagnostic subassembly at the proximal end and in communication with the diagnostic optical fiber processes diagnostic light for use in connection with a diagnostic method for detecting diseased tissue. A second fiber lumen in the catheter shaft contains a treatment optical fiber for delivering treatment light from a light source at the proximal end of the catheter shaft to the light transmission zone. The treatment optical fiber has a distal end terminating within the light transmission zone for emitting light for treatment of the diseased tissue. An occlusion balloon is positioned on the distal end of the catheter shaft adjacent to the light transmission zone. An inflation lumen in the catheter shaft and in fluid communication with the balloon delivers fluid from an inflation fluid source at the proximal end of the catheter shaft to the balloon. An infusion lumen in the catheter shaft delivers infusion fluid from an infusion fluid source at the proximal end of the catheter shaft to the distal end of the catheter shaft. One or more infusion ports formed on or near the light transmission zone and in fluid communication with the infusion lumen deliver infusion fluid to the hollow body organ, whereby blood or other opaque material can be flushed from the treatment site to provide for better diagnosis and treatment using optical methods.

### DRAWINGS

These and other features, aspects and advantages of the present invention will become more fully apparent from the following detailed description, appended claims, and accompanying drawings where:

FIG. 1A schematically illustrates the distal end of a light delivery catheter for diagnosis and treatment of diseased tissue;

FIG. 1B is a cross-sectional view of the catheter of FIG. 1A;

FIG. 2 schematically illustrates a typical optical element layout for passive IR detection;

FIG. 3 schematically illustrates a typical optical element layout for OCT imaging; and

FIG. 4 schematically illustrates a typical optical element layout for fluorescence detection or reflectance spectroscopy.

For simplicity and clarity of illustration, the drawing figures illustrate the general elements of the light delivery catheters. Description and details of well-known features and techniques are omitted to avoid unnecessarily obscuring the invention.

### DESCRIPTION

The present invention provides a catheter-based system that can be used for both diagnosis and treatment of disease conditions in body lumens, providing simultaneous or nearly simultaneous diagnosis and PDT treatment. Examples of such disease conditions include vulnerable plaques, atherosclerotic occlusions, aneurysms, cancerous lesions and abnormal vascular structures associated with cancerous conditions. The means for both diagnosis and treatment provides a significant advantage of avoiding the insertion of two catheters, one for diagnosis and a second for treatment.

The device is particularly advantageous for situations where blood elimination is desired. For example, blood elimination may be needed for effective PDT treatment as well as for optically based diagnostic technologies including optical coherence tomography (OCT), fluorescence detection (FD) and visible/IR detection. (“IR detection” is used herein to refer generally to either passive detection of IR light for optical detection of elevated temperature or for reflectance spectroscopy when either visible or IR light is used to detect changes in the reflection and transmission properties of the vessel wall.) In each of these cases the catheter provides the blood elimination means that is advantageous for both the optically based diagnostic schemes and PDT treatment.

Alternatively, diagnostic elements that do not require blood elimination could also be used with the catheter. The catheter disclosed here can be used as a combination diagnostic and treatment catheter, with the blood elimination characteristics necessary to performed the PDT treatment. Such a configuration still provides the advantage of a combining the functions of diagnosis and treatment in a single catheter. An example of such a diagnostic technology is intravascular ultrasound (IVUS).

the catheter described herein combines both the diagnostic and treatment components and also efficiently eliminates blood from the target zone, thereby improving efficacy and convenience and, in most cases, lowering overall treatment cost. A significant feature of the device is the ability to efficiently and safely eliminate blood from the target zone. The catheter can be structured around a design referred to here as an occlusion/infusion catheter. Such catheter designs are described in greater detail in U.S. patent application Ser. No. ______, entitled LIGHT DELIVERY CATHETER filed concurrently herewith, which is incorporated herein by reference in its entirety. This design can effectively remove blood from the optical light path in a manner superior to previous designs, thereby allowing for improved diagnostics and therapeutic effects. For convenience, throughout the remainder of this disclosure, the treatment shall be referred to generally as PDT, which shall include the delivery of light to the vessel wall either with or without previous administration of a photosensitive compound. Furthermore, while specific optical diagnostic technologies are provided as examples, it should be noted that the device described here is beneficial for any optically based diagnostic technology for which blood elimination provides benefit. Therefore, the scope of this disclosure is not limited solely to the specific optically-based technologies described herein.

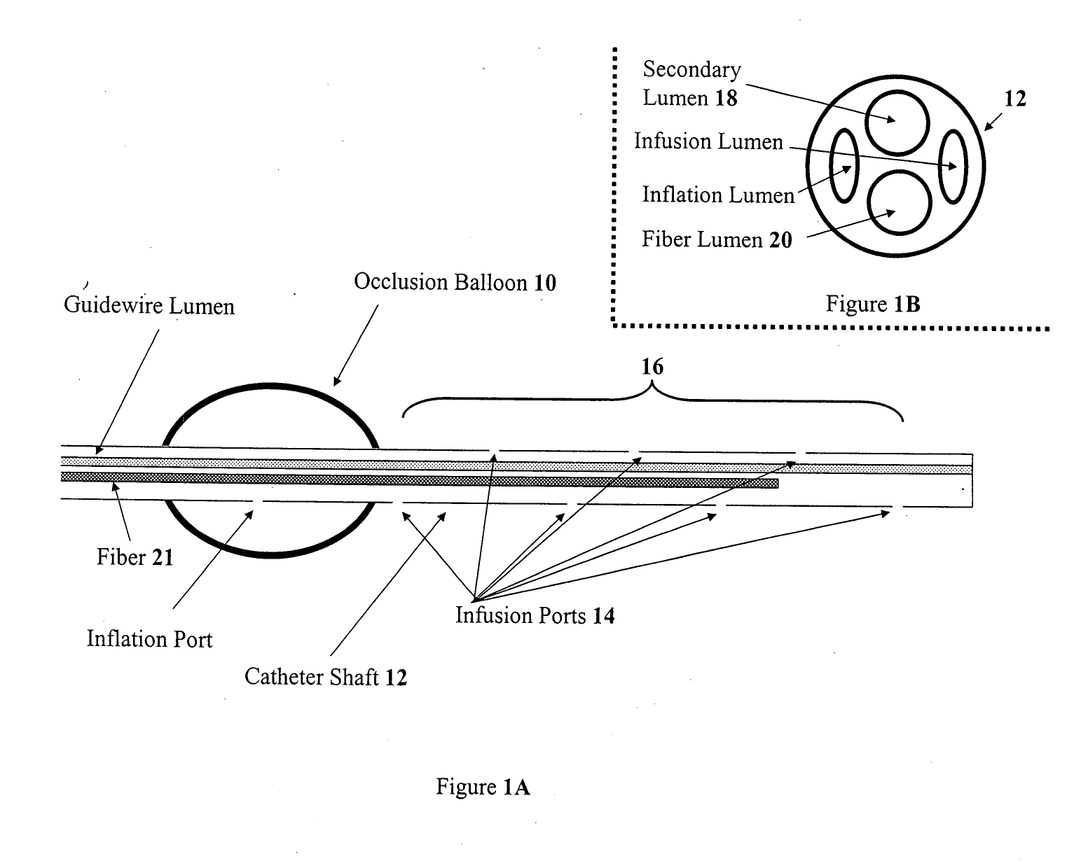

Referring to FIGS. 1A and 1B, the device preferably incorporates an occlusion balloon 10 mounted on a catheter shaft 12 such that when the occlusion balloon 10 is inflated, blood flow is blocked in the vessel. Once blood flow is blocked, a flushing fluid is injected to displace the blood adjacent to the occlusion balloon 10. Alternatively, injection of flushing fluid can be initiated prior to inflation of the occlusion balloon for convenience, as long as sufficient flush is delivered post-inflation to adequately eliminate blood. To provide optimum performance, this flushing fluid can be delivered from infusion ports 14 (or flush holes) coincident with the region of the vessel to be treated with light, which is referred to as the light transmission zone 16. If a length of vessel is to be treated, it is preferable that multiple infusion ports 14 are located around the periphery of the catheter and along the length of the light transmission zone 16. The occlusion of the vessel and infusion of flushing fluid eliminates blood to allow light to pass relatively unattenuated between the catheter shaft and the vessel wall.

The balloon 10 is positioned adjacent to the light transmission zone 16. By placing the occlusion balloon either proximal or distal of the region to receive the PDT light treatment, there is no other structure within the light transmission zone, such as a balloon, to interfere with the functioning of the diagnostic element or to disturb the tissue being diagnosed. While the device shown in FIG. 1A illustrates an occlusion balloon that is proximal to the light transmission zone, the occlusion balloon can also be positioned distal to the light transmission zone for some applications. Such a configuration may be desirable, for example, where there is insufficient space between the proximal end of the vessel and the target tissue to allow proper positioning of a proximal occlusion balloon. Alternatively the device can have occlusion balloons located both proximal and distal to the light transmission zone.

An additional advantage of this design is that elimination of the occlusion balloon from the light transmission zone allows additional features to be added in this region. For example, a temperature sensing element such as a thermocouple can be incorporated within the target zone to measure any temperature rises that result from the flushing fluid. Another example is the positioning of a temperature sensing probe designed to measure the temperature of the vessel wall.

The catheter can be positioned using a guidewire. The guidewire is first inserted within the vessel, after which the catheter is positioned by advancing it over the guidewire via secondary lumen 18. After the catheter is positioned within the vessel, the guidewire can be retracted and a separate diagnostic sensing element inserted into secondary lumen 18 and advanced to the tissue site of interest. Diagnostic elements that can be inserted in this manner include fiber-optic based diagnostic technologies such as OCT, FD visible or IR detection devices. The diagnostic element can be allowed to slide freely within the catheter such that, if desired, the diagnostic based element can be advanced distal to the light diffusing element to allow completely unobstructed optical assessment of the tissue. In such instances, it is preferable to fill any lumens within the catheter distal to the diffuser to minimize any unnecessary light reflection which may affect the diagnosis.

The device preferably includes a light delivery fiber 21, which can terminate in a light diffusing element to provide diffuse light at the light transmission zone 16. The diffusing element 22 preferably is a plastic fiber or a glass fiber with its distal tip modified to emit light in a direction substantially orthogonal to the optical axis of fiber 21. Examples of such diffuser tips are described in Doiron et al. U.S. Pat. No. 5,269,777 and Heath et al. U.S. Pat. No. 6,366,719, both of which are incorporated herein by reference in their entirety. The transparent nature of the fiber and diffuser offers minimal interference with optically based diagnostic technologies. However, it should be appreciated that the device need not include a light delivery fiber if configured solely as a diagnostic device.

A method of use of the device for diagnosis and treatment in this configuration can be summarized as follows. A guidewire is inserted in the vessel to be examined. The distal end of the catheter is then positioned within the vessel by passing it over the guidewire. The guidewire is then withdrawn and a diagnostic device is inserted into the guidewire channel of the catheter. An occlusion balloon on the catheter is then inflated to block blood flow, followed by injection of flushing fluid to clear the blood. (This step is not required prior to conducting diagnostics using IVUS.) A diagnostic procedure such as IVUS, OCT, FD and/or IR detection is then performed using the diagnostic device. After identification of the target lesion, the treatment light is turned on to deliver the PDT treatment dose. If the occlusion and flush has not been performed before the diagnostic step, the occlusion and flush is preferably performed before delivering the treatment light. If desired the diagnostic functions may continue to be monitored during treatment as a means to monitor the progress of the treatment. After treatment is complete, the catheter can be withdrawn or repositioned to identify additional treatment sites and the process is repeated as appropriate.

When using a photosentizer compound to enhance the efficacy of the treatment such as is done with PDT or when using a fluorescent compound to enhance the efficacy of the diagnosis, the compound can be introduced by either systemic administration or local delivery of drug prior to delivery of the treatment light. In the case of local delivery, the drug can be administered by the occlusion/infusion catheter. If this device is used for local drug delivery, it is preferable but not necessary to have occlusion balloons located on the catheter shaft and positioned both upstream and downstream of the infusion ports. Use of such dual balloons helps to reduce the total drug dose since they contain the drug near the treatment site.

In the case of optically based diagnostic technologies an optical signal is delivered and/or received through an optical fiber for the purposes of diagnosis. The optical signal can be transmitted using a common fiber or through separate fibers for emission and detection. Rather than terminating the fiber 21 in a diffuser, fiber 21 can be terminated in a light emitting element capable of directing light longitudinally toward the vessel wall. Light can be directed in a number of ways, for example, by polishing the fiber tip at a 45 degree angle to cause the light reaching the end of the fiber 21 to be directed normal to the axis of fiber 21. The device can be operated in either diagnostic or treatment mode, or both simultaneously. Once a target lesion has been identified, the light used for PDT treatment is passed down this same fiber 21 such that it exits the fiber at its distal end to irradiate the vessel site identified in the diagnostic step.

An advantage of this technique is that both the diagnosis and treatment light is directed at the same point on the vessel wall, minimizing any risk of missing the target lesion with the treatment wavelength or inadvertently treating an area of the vessel wall that should not receive treatment. A further advantage is that by using a common fiber for both treatment and diagnosis the overall device profile is minimized. However, separate fibers can be used for emission and detection where the emission fiber can deliver treatment light or light required for diagnosis and the detection fiber receiving the light signal necessary for diagnosis. This approach still provides the advantage of minimizing geographic mismatch since both the treatment light and diagnosis light are delivered and received within the light transmission zone. Alternatively, there could be two emission fibers, one for diagnosis and one for treatment, with a third fiber for detection, and still providing the advantage of a single treatment and detection device with minimal risk of geographic mismatch.

The catheter also allows for a lower profile device, which is advantageous in many applications. When designing a fiber based diagnostic device that can be inserted into or retracted from a catheter, the fiber is generally placed within a protective sheath to prevent damage from handling in the catheter lab. Because the diagnostic fiber can be permanently incorporated within the catheter at the time of fabrication, this sheath can be either eliminated or at least reduced in size. Alternatively, for situations where the catheter diameter is to be minimized, the separate fiber lumen and guidewire lumen can be eliminated, and replaced with a single lumen of sufficient size to allow either the guidewire or optical fiber to pass. In this way the catheter can first be positioned over the guidewire, after which the guidewire is removed and replaced with the optical fiber.

A common fiber can also be used with a short diffuser segment at the distal end of the fiber. Here the same fiber 21 is used to deliver the PDT signal and to detect the diagnostic signal. This arrangement is feasible when using the IR or FD diagnostic detection schemes. This configuration allows for a lower profile catheter, either by permanently integrating the fiber into the catheter or by eliminating the separate fiber lumen and guidewire lumen and replacing them with a single lumen. In the case of such a single lumen, the catheter is first positioned using the guidewire, after which the guidewire is retracted and replaced with the optical fiber. Alternatively, the device can be configured as a rapid exchange device as opposed to an over-the-wire device.

In the case of FD, the optical system (including the fiber in the catheter) is arranged such that light of one wavelength is directed at the diseased tissue while light of another wavelength (or range of wavelengths) emitted from the tissue is collected by the fiber such that it propagates back to the proximal end of the catheter for analysis. Typically, the emitted light, known as fluorescence, is of a longer wavelength than the incident light. The diagnosis can be performed in one of two ways. In the first case, the spectral distribution of the fluorescent light is analyzed based on the fact that fluorescence from atherosclerotic tissue has a different spectral distribution than that from healthy tissue. In the second case a fluorescent compound which accumulates differently in diseased tissue than in healthy tissue is used. This fluorescent compound is first administered to the patient, after which the diagnostic and treatment procedure is conducted. The diagnosis is conducted by moving the catheter to seek out areas that are either more strongly fluorescent than adjacent tissue (for fluorescent compounds that are more strongly fluorescent in diseased tissue than healthy tissue) or less strongly fluorescent than surrounding tissue (for fluorescent compounds which are less strongly fluorescent in diseased tissue than healthy tissue).

In the case of passive IR detection, no light is delivered to the tissue. Rather, the fiber simply collects the IR light that is being emitted by the tissue. This is a well known technique for detecting temperature changes and is promising for detecting inflamed tissues such as those associated with problematic vulnerable plaques. Inflamed tissues typically have higher temperatures than tissues that are not inflamed and therefore emit an IR spectrum that is more strongly weighted toward shorter wavelengths. In such applications it is advantageous to position a temperature sensing element, such as a thermocouple on the catheter at a position within the light treatment zone, such that temperature changes associated with flushing can be corrected.

In the case of OCT, a light source with a short coherence length is coupled to a single mode fiber such that this light can be directed at the vessel wall. Light reflected in this same wavelength range is scattered back into the fiber and transported back to the proximal end of the catheter and into an interferometer. By interfering this scattered light with a time-delayed reference beam, an image of the vessel can be constructed that is similar to that achieved with IVUS, but with significantly higher spatial resolution and, in some instances, providing complementary information to that provided by IVUS.

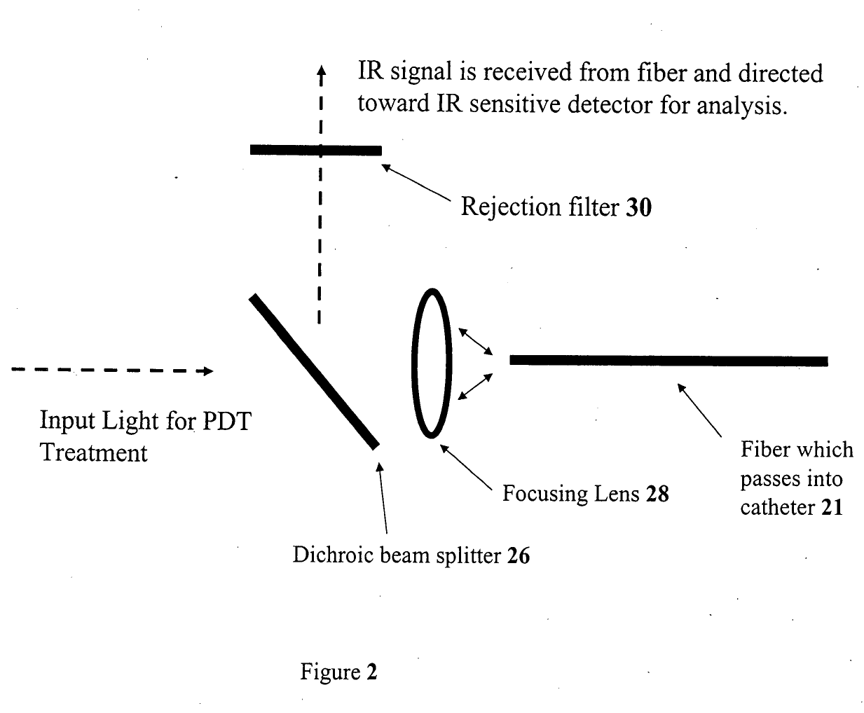

The catheter assembly preferably includes a diagnostic subassembly at the proximal end and in communication with the diagnostic optical fiber for processing diagnostic light for use in connection with a diagnostic method for detecting diseased tissue. When using a common fiber optic to send and receive optical signals for diagnostics and light for PDT treatment, the diagnostic subassembly can include optical elements for separating the diagnostic signals from the treatment light at the proximal end of the catheter. FIG. 2 illustrates a typical optical layout for separating IR and PDT wavelengths at the proximal end of the device when using a common fiber for diagnosis and treatment. A dichroic beam splitter 26 is positioned at the proximal end of the catheter. The dichroic beam splitter 26 passes short wavelength light for PDT treatment, but reflects IR light received from the fiber. Input light for PDT treatment passes through dichroic beam splitter 26 and is transmitted via focusing lens 28 into optical fiber 21. IR light received from the tissue and transmitted from the distal end of fiber 21 is collimated by focusing lens 28 and then reflected from the dichroic beam splitter 26. The reflected IR light is passed through a rejection filter 30, which allows only the IR signal to be transmitted to an IR sensitive detector or spectrometer for analysis.

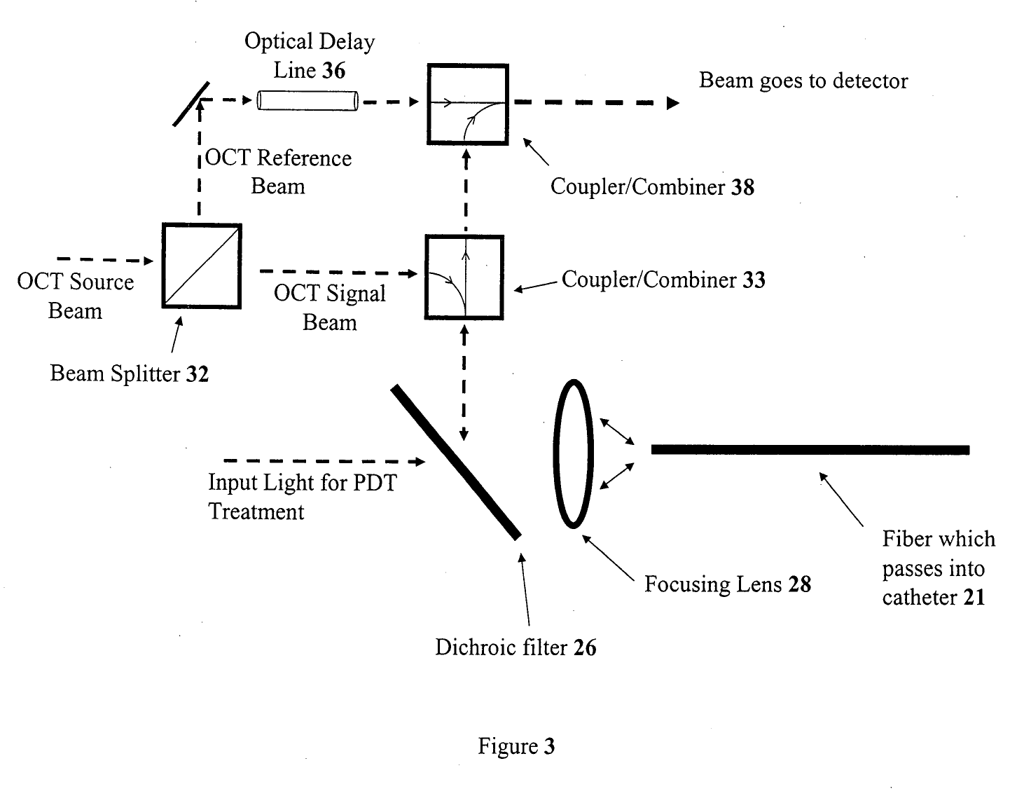

FIG. 3 illustrates a typical optical layout for separating OCT and PDT wavelengths at the proximal end of the device. A dichroic beam splitter 26 is positioned at the proximal end of the catheter. The dichroic beam splitter 26 passes short wavelength light for PDT treatment, but reflects longer wavelength OCT light received from, or directed toward, the catheter fiber 21. Input light for PDT treatment passes through dichroic beam splitter 26 and is transmitted via focusing lens 28 into optical fiber 21. The beam from the short coherence length OCT source is incident on beam splitter 32, which separates this beam into two beams, a reference beam and a signal beam. The reference beam is directed through optical delay line 36, while the signal beam is directed to fiber coupler/combiner 33 and toward dichroic beam splitter 26, from which it is reflected and focused into fiber 21 via focusing lens 28. OCT light scattered from tissue at the distal end of the catheter device is collected by the distal tip of fiber 21 and transmitted to the proximal end of fiber 21, reflected from dichroic beam splitter 26 and through fiber coupler/combiner 33. The time delayed reference beam and the beam scattered from the tissue are then combined in fiber coupler/combiner 38 into a common beam which is passed through a bandpass filter and directed to an optical detector which provides the OCT signal.

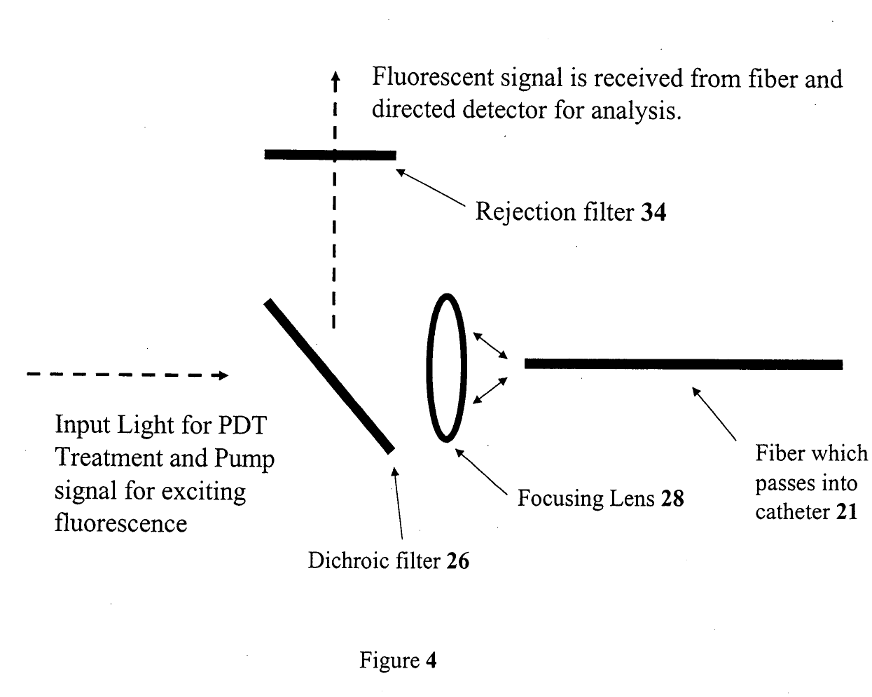

FIG. 4 illustrates a typical optical layout for separating fluorescence and PDT wavelengths at the proximal end of the device. A dichroic beam splitter 26 is positioned at the proximal end of the catheter. The dichroic beam splitter 26 passes short wavelength light for PDT treatment and also passes the short wavelength pump light that is used to excite fluorescence at the distal end of the catheter device, but reflects the longer wavelength fluorescent light. Both the PDT light and fluorescent pump light are focused by means of focusing lens 28 and directed into the fiber 21. Fluorescent light generated in the tissue as a result of pump light directed at tissue at the distal end of the catheter device is collected at the distal tip of the fiber 21 and collimated at the proximal end of the catheter device by focusing lens 28, reflected from dichroic beam splitter 26 and directed through a rejection filter 34 for analysis.

It should be noted that the optical layouts given in FIGS. 2-4 are provided by way of example. Light can be coupled into the catheter and analyzed using a number of alternative configurations. For example, in reflectance spectroscopy, a system similar to that shown in FIG. 4 could be used with the rejection filter comprising a filter that rejects light of one polarization and passes that of another.

In each of the descriptions given above, the distal end of the catheter illustrated an over-the-wire design. However, the invention is not limited to over-the-wire catheter designs but also includes rapid exchange catheter designs.

Finally, in those situations where light attenuating media such as blood are not present, the occlusion balloon and infusion ports can be eliminated if desired. Such a catheter containing both means for diagnosis and light treatment can provide convenience, reduced risk of geographical miss and lower cost.

The device can be used with any catheter-based technology, such as OCT, FD, visible/IR detection. For each of these optically based technologies, the catheter can contain an optical fiber that allows light to be transmitted between the proximal and distal ends of the catheter. Depending on the technique used, the light may be directed from the distal end to the proximal end of the catheter, from the proximal end to the distal end catheter, or both. In some cases, a range of wavelengths may be used, while in others a discrete wavelength may be used. Similarly, in some cases a single mode fiber is used whereas in others a multimode fiber is acceptable.

The catheter also provides a benefit when used with non-optical diagnostic schemes, particularly intravascular ultrasound (IVUS). While IVUS does not ordinarily require blood elimination, the catheter design presented here allows the diagnosis and PDT treatment to be performed with a single catheter, thereby avoiding the shortcomings associated with separate. diagnosis and treatment catheters identified earlier in this disclosure. The device also provides the means to introduce an index matching fluid as is often beneficial in OCT schemes.

While VP is used as an example of an indication that can be diagnosed and treated with the catheter, the device and method disclosed here are not limited to VP. Rather the device and method provide a device that may be used to diagnosis and treat a wide range of medical conditions. Examples of these include cardiovascular conditions such as atherosclerosis, restenosis, and aneurysm as well as oncologic conditions such as pre-cancerous and cancerous lesions and associated vasculature.

Although the invention has been described with reference to specific embodiments, it should be understood that various changes may be made without departing from the spirit or scope of the invention. For instance, the various features described above and shown in the drawings can be used singly or in any of various combinations. Accordingly, the disclosed examples are intended to be illustrative of the scope of the invention and are not intended to be limiting. The scope of the invention is defined as set forth in the appended claims.

We claim:

1. A catheter for detecting diseased tissue in a hollow body organ, the catheter comprising:
a. an elongated tubular catheter shaft having a proximal end which remains outside of the body organ when in use and a distal end which is inserted into the body organ when in use, the distal end having a light transmission zone through which light can be transmitted;
b. a fiber lumen in the catheter shaft for containing a diagnostic optical fiber having a distal end terminating within the light transmission zone for emitting and receiving diagnostic light through the light transmission zone;
c. a diagnostic subassembly at the proximal end and in communication with the diagnostic optical fiber for processing diagnostic light for use in connection with a diagnostic method for detecting diseased tissue;
d. an occlusion balloon positioned on the distal end of the catheter shaft adjacent to the light transmission zone;
e. an inflation lumen in the catheter shaft and in fluid communication with the balloon for delivering fluid from an inflation fluid source at the proximal end of the catheter shaft to the balloon;
f. an infusion lumen in the catheter shaft for delivering infusion fluid from an infusion fluid source at the proximal end of the catheter shaft to the distal end of the catheter shaft; and
g. one or more infusion ports formed on or near the light transmission zone and in fluid communication with the infusion lumen for delivering infusion fluid to the hollow body organ.

2. The catheter of claim 1, wherein the plurality of infusion ports are radially distributed around the circumference of the catheter shaft at the light transmission zone.

3. The catheter of claim 1, wherein the plurality of infusion ports a longitudinally distributed along the length of the light transmission zone.

4. The catheter of claim 1, wherein the diagnostic subassembly is configured for use in connection with a diagnostic method selected from the group consisting of optical coherence tomography, fluorescence detection, reflectance spectroscopy, and passive infrared detection.

5. The catheter of claim 1, wherein the diagnostic optical fiber is configured to emit light for exciting fluorescent light and to receive the fluorescent light.

6. The catheter of claim 1, wherein the diagnostic optical fiber is used to receive infrared fluorescence emitted from tissue of the hollow body organ.

7. The catheter of claim 1, wherein the diagnostic optical fiber is in communication with a light source at the proximal end of the catheter shaft and is configured to transmit treatment light to the diseased tissue via the light transmission zone.

8. The catheter of claim 1, further comprising a second fiber lumen in the catheter shaft for containing a light treatment optical fiber for delivering treatment light from a light source at the proximal end of the catheter shaft to the diseased tissue via the light transmission zone.

9. The catheter of claim 8, wherein the light treatment optical fiber has a distal end terminating in a diffuser within the light transmission zone.

10. The catheter of claim 1, further comprising a temperature sensing element for sensing temperature in the region of the light transmission zone.

11. A catheter for detecting diseased tissue in a hollow body organ, the catheter comprising:
a. an elongated tubular catheter shaft having a proximal end which remains outside of the body organ when in use and a distal end which is inserted into the body organ when in use, the distal end having a light transmission zone through which light can be transmitted;
b. a diagnostic lumen in the catheter shaft for containing a diagnostic device having a distal end terminating within the light transmission zone for capturing diagnostic information through the light transmission zone;
c. a diagnostic subassembly at the proximal end and in communication with the diagnostic device for processing the diagnostic information for use in connection with a diagnostic method for detecting diseased tissue;
d. an occlusion balloon positioned on the distal end of the catheter shaft adjacent to the light transmission zone;
e. an inflation lumen in the catheter shaft and in fluid communication with the balloon for delivering fluid from an inflation fluid source at the proximal end of the catheter shaft to the balloon;
f. an infusion lumen in the catheter shaft for delivering infusion fluid from an infusion fluid source at the proximal end of the catheter shaft to the distal end of the catheter shaft; and
g. one or more infusion ports formed on or near the light transmission zone and in fluid communication with the infusion lumen for delivering infusion fluid to the hollow body organ.

12. The catheter of claim 11, wherein the diagnostic device is an intravascular ultrasound catheter subassembly.

13. The catheter of claim 11, wherein the diagnostic device is an optical coherence tomography catheter subassembly.

14. The catheter of claim 11, wherein the diagnostic device is a fluorescence detection catheter subassembly.

15. The catheter of claim 11, wherein the diagnostic device is a catheter subassembly configured for visible or infrared light detection.

16. A catheter for detecting diseased tissue in a hollow body organ, the catheter comprising:
a. an elongated tubular catheter shaft having a proximal end which remains outside of the body organ when in use and a distal end which is inserted into the body organ when in use, the distal end having a light transmission zone through which light can be transmitted;
b. a first fiber lumen in the catheter shaft for containing a first diagnostic optical fiber having a distal end terminating within the light transmission zone for emitting diagnostic light through the light transmission zone;
c. a second fiber lumen in the catheter shaft for containing a second diagnostic optical fiber having a distal end terminating within the light transmission zone for receiving diagnostic light through the light transmission zone;
d. a diagnostic subassembly at the proximal end and in communication with the second diagnostic optical fiber for processing diagnostic light for use in connection with a diagnostic method for detecting diseased tissue;
e. an occlusion balloon positioned on the distal end of the catheter shaft adjacent to the light transmission zone;
f. an inflation lumen in the catheter shaft and in fluid communication with the balloon for delivering fluid from an inflation fluid source at the proximal end of the catheter shaft to the balloon;
g. an infusion lumen in the catheter shaft for delivering infusion fluid from an infusion fluid source at the proximal end of the catheter shaft to the distal end of the catheter shaft; and
h. one or more infusion ports formed on or near the light transmission zone and in fluid communication with the infusion lumen for delivering infusion fluid to the hollow body organ.

17. A catheter for detecting diseased tissue in a hollow body organ, the catheter comprising:
a. an elongated tubular catheter shaft having a proximal end which remains outside of the body organ when in use and a distal end which is inserted into the body organ when in use, the distal end having a light transmission zone through which light can be transmitted;
b. a fiber lumen in the catheter shaft for containing a diagnostic optical fiber having a distal end terminating within the light transmission zone .for receiving diagnostic light through the light transmission zone;
c. a diagnostic subassembly at the proximal end and in communication with the diagnostic optical fiber for processing diagnostic light for use in connection with a diagnostic method for detecting diseased tissue;
d. an occlusion balloon positioned on the distal end of the catheter shaft adjacent to the light transmission zone;
e. an inflation lumen in the catheter shaft and in fluid communication with the balloon for delivering fluid from an inflation fluid source at the proximal end of the catheter shaft to the balloon;
f. an infusion lumen in the catheter shaft for delivering infusion fluid from an infusion fluid source at the proximal end of the catheter shaft to the distal end of the catheter shaft; and
g. one or more infusion ports formed on or near the light transmission zone and in fluid communication with the infusion lumen for delivering infusion fluid to the hollow body organ.

18. A catheter for detecting and treating diseased tissue in a hollow body organ, the catheter comprising:
a. an elongated tubular catheter shaft having a proximal end which remains outside of the body organ when in use and a distal end which is inserted into the body organ when in use, the distal end having a light transmission zone through which light can be transmitted;
b. a first fiber lumen in the catheter shaft containing a diagnostic optical fiber having a distal end terminating within the light transmission zone for emitting and receiving light through the light transmission zone;
c. a diagnostic subassembly at the proximal end and in communication with the diagnostic optical fiber for processing diagnostic light for use in connection with a diagnostic method for detecting diseased tissue;
d. a second fiber lumen in the catheter shaft for containing a treatment optical fiber for delivering treatment light from a light source at the proximal end of the catheter shaft to the light transmission zone, the treatment optical fiber having a distal end terminating within the light transmission zone for emitting light for treatment of the diseased tissue;
e. an occlusion balloon positioned on the distal end of the catheter shaft adjacent to the light transmission zone;
f. an inflation lumen in the catheter shaft and in fluid communication with the balloon for delivering fluid from an inflation fluid source at the proximal end of the catheter shaft to the balloon;
g. an infusion lumen in the catheter shaft for delivering infusion fluid from an infusion fluid source at the proximal end of the catheter shaft to the distal end of the catheter shaft; and
h. one or more infusion ports formed on or near the light transmission zone and in fluid communication with the infusion lumen for delivering infusion fluid to the hollow body organ.

19. The catheter of claim 18, wherein the plurality of infusion ports are radially distributed around the circumference of the catheter shaft at the light transmission zone.

20. The catheter of claim 18, wherein the plurality of infusion ports a longitudinally distributed along the length of the light transmission zone.

21. The catheter of claim 18, wherein the diagnostic optical fiber is configured for use in connection with a diagnostic method selected from the group consisting of optical coherence tomography, fluorescence detection, reflectance spectroscopy, and passive infrared detection.

22. The catheter of claim 18, wherein the diagnostic optical fiber comprises an optical fiber configured to emit light of exciting fluorescent light and to receive the fluorescent light.

23. The catheter of claim 18, wherein the diagnostic optical fiber is used to receive infrared fluorescence emitted from tissue of the hollow body organ.

24. The catheter of claim 18, wherein the diagnostic subassembly further comprises a wavelength selective optical element at the proximal end of the one or more optical fibers to filter light received through the one or more optical fibers.

25. The catheter of claim 18, further comprising a temperature sensing element for sensing temperature in the region of the light transmission zone.
